(19) United States
(12) Patent Application Publication
(10) Pub. No.: US 2025/0376396 A1
(43) Pub. Date: Dec. 11, 2025

(54) METHOD FOR REMOVING EXTRACELLULAR ANTIBIOTIC RESISTANCE GENES IN WATER

(71) Applicant: NANJING AGRICULTURAL UNIVERSITY, Nanjing (CN)

(72) Inventors: Guanyu ZHENG, Nanjing (CN); Fangjuan LI, Nanjing (CN); Lixiang ZHOU, Nanjing (CN)

(21) Appl. No.: 18/874,043

(22) PCT Filed: Jun. 13, 2023
(86) PCT No.: PCT/CN2023/099820

§ 371 Date: Dec. 11, 2024

(30) Foreign Application Priority Data
Sep. 20, 2022 (CN) 202211148443.6

(51) Int. Cl. C02F 3/34; C02F 101/34; C02F 101/38; C12N 11/087

(52) U.S. Cl. CPC C02F 3/342; C02F 3/348; C12N 11/087; C02F 2101/34; C02F 2101/38

(57) ABSTRACT

Disclosed is a method for removing extracellular antibiotic resistance genes in water, which belongs to the field of environmental management. The method comprises adding a cell lysate, especially a cell lysate of E. coli, to water, wherein the cell lysate comprises various enzymes such as ribonucleotide exonuclease I, ribonucleotide exonuclease III, and DNA topoisomerase I, and can degrade extracellular antibiotic resistance genes. In addition, in view of the problems of poor stability of the cell lysate in an aqueous solution and the enzymes in the cell lysate being easily deactived due to complicated water ingredients, the present invention provides a cell lysate immobilized with a polyacrylamide (PAM) hydrogel, such that extracellular antibiotic resistance genes in sewage can be effectively reduced and the cell lysate can be reused by means of protecting the cell lysate.

### TECHNICAL FIELD

The present invention belongs to the technical field of environmental management, and specifically relates to a method for removing extracellular antibiotic resistance genes in water.

### BACKGROUND

Antibiotics are widely used in medicine, aquaculture and animal husbandry, and greatly contribute to human health and agricultural production. However, the overuse and abuse of antibiotics inevitably causes unused antibiotics to enter the environment, inducing and accelerating the generation of Antibiotic resistant bacteria (ARB) and Antibiotic resistance genes (ARGs). Antimicrobial resistance (AR) is becoming increasingly serious. The World Health Organization (WHO) has emphasized that antimicrobial resistance is complex and multi-faceted, and is one of the major threats to the development of human society. It was not until 2006 that ARGs were recognized as an emerging environmental pollutant.

ARGs mainly exist in two forms in the environment: intracellular DNA and extracellular DNA. Intracellular antibiotic resistance genes (iARGs) mainly exist in chromosomes or Mobile genetic elements (MEGs) in ARB. Extracellular antibiotic resistance genes (eARGs) generally exist in MEGs in the environment or are exposed to environmental media. iARGs promote the proliferation of ARB by means of conjunction and transduction, while eARGs are often absorbed by bacteria through transformation so as to promote the spread of antibiotic resistance genes.

The risk of eARGs is becoming increasingly significant at present, and eARGs have been detected in a variety of environments. It is reported that the abundance of eARGs in livestock and poultry manure is 1.7×10³ to 4.2×10⁸ copies/g dry sludge. Although iARGs in sludge can be effectively removed by various conditioning methods (such as bioleaching), the abundance of eARGs such as aminoglycosides and tetracyclines is still as high as 10⁷ copies/L. Similarly, the content of eDNA detected in effluent which is treated by membrane bioreactors in wastewater treatment plants is 4.2 ng/mL. Disinfection is an important unit to remove pollutants so as to improve the quality of secondary effluent in wastewater treatment plants, but some studies have found that the abundance of eARGs shows an increasing trend after disinfection.

A large number of studies have assessed the removal of ARGs in water by different methods, mainly involving physical methods (such as coagulation), chemical methods (such as oxidation, Fenton, and photocatalysis), physical-chemical combined methods (such as UV radiation and ionizing radiation) and biological methods (such as membrane bioreactors and constructed wetlands). Some coagulants, such as polyaluminum chloride, can effectively remove ARGs, but the removal efficiency is significantly correlated to the administration dosage of coagulants, and the cost is high, therefore, coagulants are not economically feasible. For chemical treatment such as ozone oxidation, the relative abundance of vanA and blaVIM increases after O₃ treatment, and the treatment effect of O₃ disinfection on ARGs is affected by various factors such as ozone concentration, contact time, pH, suspended matter, and humic acid concentration. Due to its limited mass transfer rate and low water solubility, the actual utilization rate of O₃ is not high. Some light-mediated advanced oxidation can effectively remove ARGs, for example, UV/H₂O₂, UV/TiO₂, photocatalysis, and photo-Fenton achieve the removal of ARGs being 0.83 to 5.59 logs, but the cost thereof is high. UV-based photocatalysis has high energy consumption, and the turbidity of wastewater inhibits UV penetration. The process of UV disinfection does not produce disinfection byproducts and is friendly to the environment, but some studies have shown that a conventional UV disinfection dose has no removal effect on various antibiotic resistance genes against antibiotics such as tetracyclines, sulfonamides and erythromycins. The removal efficiency of ARGs by membrane bioreactors and constructed wetlands is limited by operating parameters, including hydraulic retention time and hydraulic loading, and the concentration of soluble microbial products and extracellular polymers in the polluted membrane has a great influence on the removal of ARGs. In the treatment by constructed wetland, plant species, planting patterns and wetland types have a certain impact. Although there are already many techniques that can relieve the spread of ARGs to some extent at present, the limitations of each technique are self-evident. Moreover, most studies mainly focus on existing treatment techniques, without more attention to technological innovation, and there are few biological methods to remove eARGs.

There are many applications of natural enzymes produced by microorganisms in the degradation of pollutants. Some studies have found that laccase produced by Bjerkandera adusta TBB-03 can effectively degrade persistent pollutional drugs in water environment. Similarly, sludge microbial cell lysates contain a variety of natural enzymes, such as acid phosphatase, beta-galactosidase and beta-glucosidase, and oxidoreductase, which can catalytically degrade micropollutants (including antibiotics). There are various species of enzymes produced by bacteria, and nuclease is one of the most important ones, and its species are abundant, including endonuclease I and the like, which can catalytically degrade DNA. However, the free enzyme aqueous solution has a poor environmental stability, therefore, immobilization is an effective means to achieve its reuse and improved stability.

### SUMMARY OF THE INVENTION

### 1. Objectives of the Invention

In view of the problems existing in the existing methods for removing eARGs in water, the present invention provides a method for removing extracellular antibiotic resistance genes in water. The method comprises adding a cell lysate, especially a cell lysate of E. coli MG1655 (WT), to water, wherein the cell lysate comprises various enzymes such as exonuclease I, exonuclease III, and DNA topoisomerase I, and can degrade extracellular antibiotic resistance genes. In addition, in view of the problems of poor stability of the cell lysate in an aqueous solution and the enzymes in the cell lysate being easily deactived due to complicated ingredients in water, the present invention provides a cell lysate immobilized with a polyacrylamide (PAM) hydrogel, such that extracellular antibiotic resistance genes in sewage can be effectively reduced and the cell lysate can be reused by means of protecting the cell lysate.

### 2. Technical Solutions

To solve the above problems, the technical solutions used in the present invention is as follows.

The present invention provides a method for removing extracellular antibiotic resistance genes in water, wherein the method comprises adding a cell lysate of bacteria, and the cell lysate described above contains enzymes capable of degrading DNA and can degrade eARGs.

Preferably, the bacteria include E. coli, Acinetobacter sp., Pseudomonas putida and/or Acidithiobacillus ferrooxidans.

Preferably, the bacteria include E. coli.

Preferably, the E. coli is E. coli MG1655 (WT).

Preferably, the Acinetobacter sp. is Acinetobacter baylyi ADP1.

Preferably, the Pseudomonas putida is Pseudomonas putida KT2440.

Preferably, the Acidithiobacillus ferrooxidans is Acidithiobacillus ferrooxidans LX5, and its preservation number is CGMCC NO.0727, preservation time: Mar. 13, 2002, classification and nomenclature: Thiobacillus, Thiobacillus ferrooxidans, preservation unit: China General Microbiological Culture Collection Center, preservation address: Institute of Microbiology, Chinese Academy of Sciences, Datun Road, Chaoyang District, Beijing, see Chinese invention patent with publication number CN 1375553A for details.

Preferably, the method for disrupting cells is ultrasonic disruption.

Preferably, the conditions for ultrasonic disruption are as follows: instrument parameters set to the power of 10% to 40%, the temperature of 0° C. to 4° C., the interval of 5.0 s to 9.9 s per running of 3.0 s to 5.0 s, and the ultrasonic time of 5 min to 10 min.

Preferably, the cells are cultured to a density of about 10⁹ cells/mL prior to disruption.

Preferably, after the cells are disrupted, the suspension of the disrupted bacteria is centrifuged for 10 min at 4° C. at 10000×g, and then the supernatant is filtered with a 0.22 μm sterilized syringe to remove residual cells or cell debris, and then a cell lysate is obtained.

Preferably, the cell lysate is immobilized by hydrogel and then added to water. In view of the problems of poor stability of the cell lysate in an aqueous solution and the enzymes in the cell lysate being easily deactived due to complicated water ingredients, the present invention provides a cell lysate immobilized with a hydrogel, such that extracellular antibiotic resistance genes in sewage can be effectively reduced by means of protecting the cell lysate, and reuse can be achieved.

Preferably, the hydrogel is polyacrylamide (PAM) hydrogel.

Preferably, the method for immobilizing with a hydrogel comprises obtaining the cell lysate, adding acrylamide, N,N′-bisacrylamide and K₂S₂O₈ to the cell lysate, using N₂ to aerate the mixed solution and then performing the synthesis of the hydrogel.

Preferably, acrylamide, N,N′-bisacrylamide and K₂S₂O₈ are added to the cell lysate, and N₂ is used to aerate the mixed solution for 20 min. Meanwhile, the solution was homogenized by stirring with a magnetic stirrer. The beaker is placed in a 37° C. water bath for about 30 min and then the hydrogel is synthesized.

Preferably, the temperature of the water is 28° C. to 37° C.

The present invention also provides a microbial agent comprising the cell lysate or the immobilized cell lysate.

The present invention also provides the use of the microbial agent in the treatment of wastewater containing extracellular antibiotic resistance genes.

### 3. Benificial Effects

Compared with the prior art, the present invention has the following beneficial effects.

(1) The method for removing extracellular antibiotic resistance genes in water provided in the present invention can degrade eARGs by adding the cell lysate of bacteria E. coli MG1655, Acinetobacter baylyi ADP1, Pseudomonas putida KT2440 and Acidithiobacillus ferrooxidans LX5, effectively reduce the abundance of ARGs and decrease its transformation frequency, and greatly reduce the transmission risk of eARGs in a relatively short time.

(2) The method for removing extracellular antibiotic resistance genes in water provided in the present invention can provide mild, simple and easy reaction conditions, a high removal rate and a short treatment time, and the method has no deterioration effect on water quality, is friendly to the environmental, has low treatment cost, and has a good application prospect.

(3) The method for removing extracellular antibiotic resistance genes in water provided in the present invention degrades ARGs in sewage by immobilizing enzymes in the cell lysate. Due to the poor water solubility, poor environmental stability and difficult recycling of the cell lysate, and complicated sewage ingredients, the cell lysate is immobilized. The immobilized cell lysate achieves reuse and stability improvement, and has a better treatment effect on ARGs in sewage. In addition, the following disadvantages are avoided: the effect of photocatalytic degradation of ARGs in sewage is affected by water turbidity, or disinfection treatment produces disinfection byproducts.

(4) The method for removing extracellular antibiotic resistance genes in water provided in the present invention uses natural enzymes in the cell lysate to treat micropollutants in water. Although the industrially purified enzymes can effectively degrade pollutants, the preparation process thereof is cumbersome and expensive, and therefore the industrially purified enzymes are not economically feasible.

### BRIEF DESCRIPTION OF THE DRAWINGS

FIG. 1 shows the effect of cell lysates of different bacterial strains in pure water on the reduction of eARGs.

FIG. 2 shows the effect of different concentrations of E. coli MG1655 (WT) cell lysates on the reduction of eARGs.

FIG. 3 shows the effect of E. coli MG1655 (WT) cell lysate on the reduction of eARGs at room temperature.

FIG. 4 shows the characterization of the polyacrylamide (PAM) hydrogel immobilized cell lysate.

FIG. 5 shows the effect of polyacrylamide hydrogel immobilized E. coli MG1655 (WT) cell lysate on the degradation of eARGs in sewage.

### DETAILED DESCRIPTION

The present invention is further described in conjunction with specific examples below.

It is needed to illustrate that the terms quoted in the present description, such as “on”, “below”, “left”, “right”, “middle”, etc., are only for the sake of clarity, without limiting the implementable scope, and changes or adjustments in their relative relations shall also be considered in the implementable scope of the present invention, without substantial changes in the technical content.

Unless otherwise defined, all technical and scientific terms used herein have the same meaning as those generally understood by a person skilled in the technical field of the present invention; the term “and/or” as used herein includes any and all combinations of one or more related listed items.

Examples where the specific conditions are not indicated are carried out according to the routine conditions or those recommended by the manufacturer. Reagents or instruments used, whose manufacturers are not indicated, are conventional products that can be purchased commercially.

As used herein, the term “about” is used to provide the flexibility and inaccuracy associated with a given term, measure, or value. Those skilled in the art can readily determine the degree of flexibility of specific variables.

As used herein, the term “at least one of . . . ” is intended to be synonymous with “one or more of . . . ”. For example, “at least one of A, B, and C” explicitly includes only A, only B, only C, and their respective combinations.

Concentrations, quantities, and other numerical data herein can be presented in range format. It should be understood that such a range format is used for convenience and brevity only, and should be interpreted flexibly to include not only the numerical values explicitly stated as the limits of the range, but also all individual numerical values or subranges that are encompassed in the range, as if each numerical value and subrange are explicitly stated. For example, the numerical value range from about 1 to about 4.5 should be interpreted to include not only explicitly stated limit values from 1 to about 4.5, but also individual numbers (such as 2, 3, 4) and subranges (such as 1 to 3, 2 to 4, etc.). The same principle applies to ranges that state one numerical value only, such as “less than about 4.5”, which should be interpreted to include all of the above values and ranges. Moreover, this interpretation should apply regardless of the breadth of scopes or features described.

Any of the steps described in any of method or process claims may be performed in any order and are not limited to the order set forth in the claims.

### EXAMPLE 1

This example provided a method for degrading eARGs in pure water with cell lysates, specifically including the following steps:

(1) Escherichia coil MG1655 (WT), Acinetobacter baylyi ADP1 and Pseudomonas putida KT2440 preserved at −20° C. were respectively inoculated into sterilized 50 mL of LB medium, and the medium was placed in a shaker at 37° C. and at 120 rpm and cultured for a certain time (12 h) until the bacterial density was about 10⁹ cells/mL, for subsequent experiments, wherein Escherichia coil MG1655 (WT), Acinetobacter baylyi ADP1, and Pseudomonas putida KT2440 were purchased from Beijing Biobw Biotechnology Co., LTD.

A suitable amount of Acidithiobacillus ferrooxidans LX5 was inoculated into 100 mL of modified 9K liquid medium (pH 2.50), and FeSO₄.7H₂O was added at a concentration of 44.2 g/L; 0.1 M sulfuric acid was used to adjust the pH to about 2.50, and then the medium was placed in a shaker at 28° C. and at 180 rpm and cultured for 72 h until there was no residual ferrous ions; and the medium was filtered by a qualitative medium speed filter paper to obtain the bacterial suspension of A.ferrooxidans LX5. The bacterial density was 7.12×10⁷ cells/mL by microscopic counting. In order to maintain the same density as other bacteria, 56 mL of the bacterial suspension was centrifuged at 8000×g for 10 min, the supernatant was discarded, and 2 mL of 9K medium was added for resuspension to achieve a bacterial density of 2×10⁹ cells/mL.

(2) 20 mL of each bacterial suspension cultured in step (1) was centrifuged at 8000×g for 10 min to remove the medium; Tris-HCl (pH 7.1, 10 mM) was added for resuspension, the suspension was centrifuged at 8000 g for 10 min and the supernatant was discarded, and this step was repeated once; and then the bacterial suspension was obtained by using 20 mL Tris-HCl (pH 7.1, 10 mM) for resuspension.

(3) each bacterial suspension in step (2) was subjected to ultrasonic disruption, wherein the instrument parameters were set to the power of 135 W/mL, the temperature of 4° C., the interval of 9.9 s per running of 3.0 s, and the ultrasonic time of 7 min, and the tube of each bacterial suspension was kept in ice bath throughout the ultrasonic disruption.

(4) the suspension of the disrupted bacteria is centrifuged for 10 min at 4° C. at 10000×g, and then the supernatant is filtered with a 0.22 μm sterilized syringe to remove residual cells or cell debris, and then a cell lysate is obtained; the cell lysate was deactived by heating at 95° C. for 20 min and acted as a negative control, and the supernatant was taken and stored at 4° C. for later use.

(5) 500 μL of each cell lysate of each bacterium prepared in step (4) was taken into a series of 2 mL centrifuge tubes, pUC19 plasmid (as a model eARG) was added into each centrifuge tube to its final concentration of 0.1 ng/μL, and the vortex was performed to mix well; parallel experiments were set in triplicates in all experimental groups; the centrifuge tubes were placed in a light-tight incubator at 28 to 37° C. constant temperature, and the concentration of eARG was determined by sampling and measuring the system at 24 h and 48 h, respectively.

(6) a PCR product purification kit was used to purify the sample in step (5), referring to its instructions for operation steps, such that the protein and other ingredients in the sample were removed. The purified DNA sample was stored at −20° C. for determination.

(7) qPCR quantitative experiment: a QuantStudio™ 6 and 7 Flex real-time fluorescent quantitative PCR system was used to quantitatively determine the amp^{R} gene contained in pUC19 in the DNA sample obtained in step (6); the reaction system for qPCR (10 μL) comprised 5 μL of SYBR Green I dye, 0.5 μL of a forward primer, 0.5 μL of a reverse primer, 2 μL of ddH₂O and 2 μL of the sample; and the primers for qPCR were respectively: amp^{R} F: 5′-CTATGTGGCGCGGTATTATCC-3′; amp^{R} R: 3′-CCGCAGTGTTATCACTCATG-5′. qPCR reaction procedure: predenaturation at 95° C. for 10 min; in each cycle, denaturation at 95° C. for 30 s, anneal at 56° C. for 15 s, extension at 70° C. for 15 s, a total of 40 cycles.

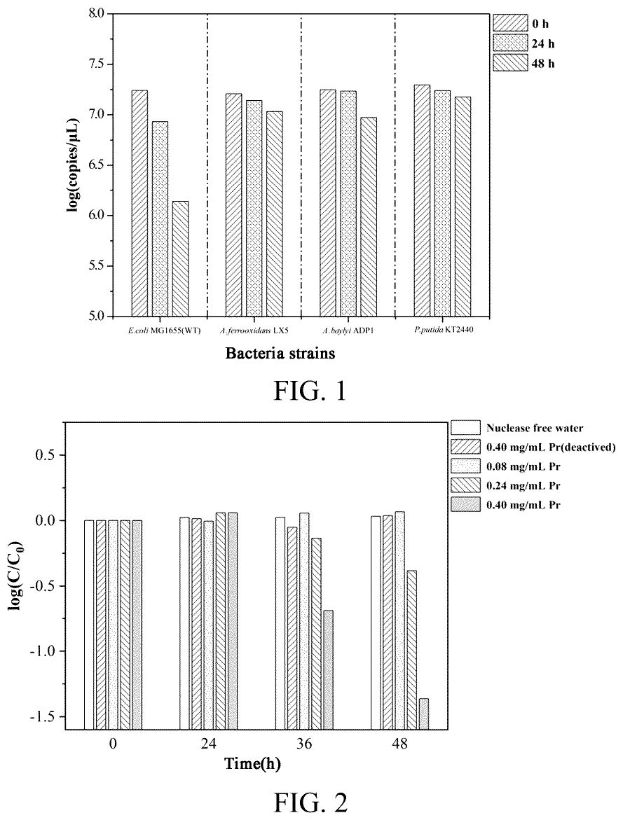

Analysis of results: the results are as shown in FIG. 1, wherein the cell lysates of bacteria E. coil MG1655 (WT) and A. baylyi ADP1, Pseudomonas putida KT2440 and Acidithiobacillus ferrooxidans LX5 can all degrade eARGs, and E. coli MG1655 (WT) cell lysate shows better treatment results.

### EXAMPLE 2

This example provided a method for degrading eARGs in pure water with different concentrations of E. coli MG1655 (WT) cell lysates, wherein the basic steps were the same as those in example 1, except that the concentrations of E. coli MG1655 (WT) cell lysates were controlled in step (5), and the ratios of the cell lysates to sterile water were 1:0, 3:2, 1:4 and 0:1, respectively. The final protein concentrations in each treatment group were respectively 0.4 mg/mL, 0.24 mg/mL, 0.08 mg/mL and 0 mg/mL, and were recorded as 0.4 mg/mL Pr, 0.24mg/mL Pr, 0.08 mg/mL Pr and 0 mg/mL Pr, respectively. Moreover, a group of an undiluted cell lysate was heated in a 95° C. water bath for 20 min to inactivate the protein therein, which was used as an deactived treatment group, and recorded as 0.4 mg/mL Pr (deactived).

The results are as shown in FIG. 2, wherein the higher the concentration of E. coli MG1655 (WT) cell lysate, the more obvious the effect of the degradation of eARGs.

### COMPARATIVE EXAMPLE

This comparative example provided a method for degrading eARGs with E. coli MG1655 (WT) cell lysates in pure water, wherein the basic steps were the same as those in example 1, except that the temperature of E. coli MG1655 (WT) cell lysates were controlled at 25° C. in step (5).

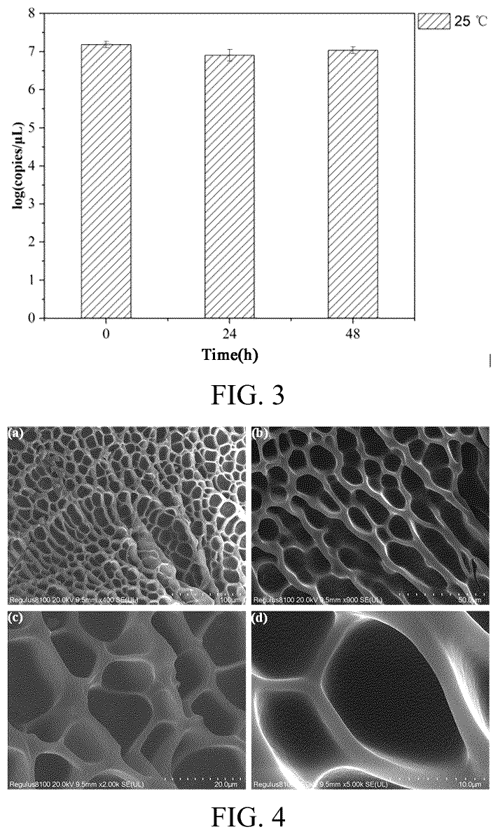

The results are as shown in FIG. 3, wherein E. coli MG1655 (WT) cell lysates have little effect on the degradation of eARGs at 25° C.

### EXAMPLE 3

This example provided protein composition analysis of the E. coli MG1655 (WT) cell lysate. The protein species in the E. coli MG1655 (WT) cell lysate were determined by two-dimensional liquid chromatography-linear ion trap-orbitrap mass spectrometer, and the primary and secondary mass spectrometry data were compared with the database and analyzed. Data results showed that the cell lysate contained 1124 kinds of proteins, among which there were 11 kinds of enzymes that could catalyze DNA degradation, including exoribonucleases and DNA topoisomerases, as shown in Table 1. The catalytic active sites of each enzyme were determined by searching in UniProt database, and the results are as shown in Table 2.

TABLE 1
Species of nucleases in cell lysates

| | | | | Gene |
|---|---|---|---|---|
| NO. | Database | Protein IDs | Protein Names | names |
| 1 | UniProt | Q2A0K9; Q2A0L0; P04995; | Exodeoxyribonuclease I | sbcB |
| | | A0A6D2W0T3; A0A0G3HG9 | | |
| 2 | UniProt | A0A4S4Z266; P09030 | Exodeoxyribonuclease III | xthA |
| 3 | UniProt | A0A4S5B0R9; P67095 | Phosphodiesterase YfcE | YfcE |
| 4 | UniProt | A0A4S5ARE1; P0A8G9; | Exodeoxyribonuclease 7 small | xseB |
| | | C4ZTH9; B1XF10 | subunit | |
| 5 | UniProt | A0A6D2VXY0; P04994; | Exodeoxyribonuclease 7 large | xseA |
| | | C4ZX84; B1XAY4 | subunit | |
| 6 | UniProt | A0A4S5APX0; P06612 | DNA topoisomerase I | topA |
| 7 | UniProt | A0A4S5AYG2; P20083 | DNA topoisomerase 4 subunit B | parE |
| 8 | UniProt | A0A4S5B2P7; P0AFI2 | DNA topoisomerase 4 subunit A | parC |
| 9 | UniProt | A0A6D2W604; P12295; | Uracil-DNA glycosylase | ung |
| | | C4ZYK3; B1XBQ7 | | |
| 10 | UniProt | A0A6D2XWL9; P0A9H1; | G/U mismatch-specific DNA | mug |
| | | C4ZQY5; B1XG73 | glycosylase | |
| 11 | UniProt | P0AES6; A0A4S5B230 | DNA gyrase subunit B | gyrB |

TABLE 2
Catalytic active sites of nucleases in cell lysates

| NO. | Enzyme names | Function |
|---|---|---|
| 1 | Exonuclease I (EC 3.1.11.1) | Digest ssDNA in the 3′→5′ direction to |
| | | generate nucleoside-5′-phosphate |
| 2 | Exonuclease III (EC 3.1.11.2) | Digest ssDNA in the 3′→5′ direction to |
| | | generate nucleoside-5′-phosphate |
| 3 | Phosphodiesterase YfcE (EC 3.1.1.4) | Hydrolyze phosphodiester bonds in various |
| | | natural substrates (cAMP, cGMP, single- or |
| | | double-stranded DNA, RNA, and |
| | | phospholipids) |
| 4 | Exodeoxyribonuclease 7 small subunit | Catalyze the cleavage of nucleosides in the |
| | (EC 3.1.11.6) | 5′→3′ or 3′→5′ direction to generate |
| | | nucleoside 5′-phosphate |
| 5 | Exodeoxyribonuclease 7 large subunit | Catalyze the cleavage of nucleosides in the |
| | (EC 3.1.11.6) | 5′→3′ or 3′→5′ direction to generate |
| | | nucleoside 5′-phosphate |
| 6 | DNA topoisomerase I (EC 5.6.2.1) | Catalyze the breaking and binding of DNA |
| | | strands, control the topological state of |
| | | DNA, independent of ATP |
| 7 | DNA topoisomerase 4 subunit B (EC | Catalyze the breaking and binding of DNA |
| | 5.6.2.2) | strands, dependent of ATP |
| 8 | DNA topoisomerase 4 subunit A (EC | Catalyze the breaking and binding of DNA |
| | 5.6.2.2) | strands, dependent of ATP |
| 9 | Uracil-DNA glycosidase (EC 3.2.2.27) | Hydrolyze single-stranded DNA or |
| | | mismatched double-stranded DNA and |
| | | polynucleotides, release free uracil |
| 10 | G/U mismatch-specific DNA | Specifically hydrolyze mismatched double- |
| | glycosylase (EC 3.2.2.28) | stranded DNA and polynucleotides, release |
| | | free uracil |
| 11 | DNA gyrase subunit B (EC 5.6.2.2) | Catalyze the breaking and binding of DNA |
| | | strands, dependent of ATP |

### EXAMPLE 4

The example provided a method for degrading eARGs in sewage with polyacrylamide (PAM) hydrogel immobilized cell lysates, specifically including the following steps:

(1) preparation of the material of polyacrylamide (PAM) hydrogel immobilized cell lysate: the preparation of the cell lysate is the same as that in example 1, except that the volume of Tris-HCl used for resuspension was 1/10 of the initial bacterial suspension volume; 20 mL of the cell lysate was put into a beaker, 1.065 g of acrylamide, 0.1 g of N,N′-methylene bisacrylamide, 0.122 g of K₂S₂O₈ were added to the beaker, and the solution was stirred with a magnetic agitator for 20 min under the purge of N₂ to achieve homogenization; after that, under the protection of N₂, the solution was heated in a water bath at 37° C. for 30 min to achieve completely gelation of the solution; the fresh gel was immersed in ultra-pure water for 24 h to remove unreacted monomers and initiators; and the gel was washed in ultra-pure water for 3 times, and then dried in a freeze dryer. After drying, it was stored in a refrigerator at −20° C. for subsequent experiments.

(2) the cell lysate was heated at 95° C. for 20 min to deactive the enzymes therein, and it was used as a negative control for synthesis of the polyacrylamide hydrogel immobilized material. The other steps were the same as those in step (1).

(3) 0.15 g of materials prepared in steps (1) and (2) were weighed respectively and dissolved into 30 mL of sewage, with the concentration being 5 mg/L (equivalent to 0.40 mg/mL of protein content); pUC19 plasmid was added to achieve the final concentration of 0.001 ng/μL (pUC19 was selected as a model eARG); the mixture was cultured in a shaker at 37° C. and at 180 rpm; parallel experiments were set in triplicates in all experimental groups; after 24 h of reaction, after standing, the supernatant was separated and eARGs and TOC were determined; 30 mL of sewage containing 0.001 ng/μL of pUC19 plasmid continued to be added to the system, and started the next cycle of degradation experiment, and the procedure was repeated twice.

(4) 44 mL of anhydrous ethanol and 2 mL of 3 M sodium acetate were added to 20 mL of the sample in step (3); the mixture was stored at −20° C. overnight, and then centrifuged at 10000×g for 10 min to precipitate DNA; the supernatant was discarded, 10 mL of 1×PBS was added to resuspend DNA precipitates, and then freeze drying was performed. DNA was extracted from the freeze-dried sample using the DNeasy® PowerSoil® Pro DNA extraction kit (QIAGEN, Germany). For the operation steps, instructions of the kit were referred to, and the extracted DNA sample was stored at −20° C. for future use.

(5) qPCR quantitative experiment: a QuantStudio™ 6 and 7 Flex real-time fluorescent quantitative PCR system was used to quantitatively determine the amp^{R} gene contained in pUC19 in the DNA sample obtained in step (4); the reaction system for qPCR (10 μL) comprised 5 μL of SYBR Green I dye, 0.5 μL of a forwardprimer, 0.5 μL of a reverse primer, 2 μL of ddH₂O and 2 μL of the sample; and the primers for qPCR were respectively: amp^{R} F: 5′-CTATGTGGCGCGGTATTATCC-3′; amp^{R} R: 3′-CCGCAGTGTTATCACTCATG-5′. qPCR reaction procedure: predenaturation at 95° C. for 10 min; in each cycle, denaturation at 95° C. for 30 s, anneal at 56° C. for 15 s, extension at 70° C. for 15 s, a total of 40 cycles.

### Analysis of Results

The material of polyacrylamide (PAM) hydrogel immobilized cell lysate is as shown in FIG. 4.

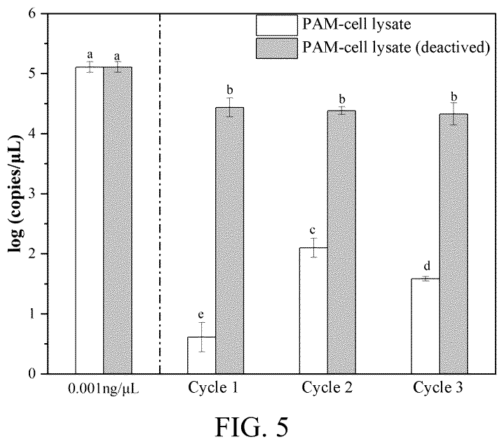

The results of the reduction of eARGs in sewage by the polyacrylamide hydrogel immobilized cell lysate are as shown in FIG. 5. The polyacrylamide hydrogel immobilized cell lysate can effectively reduce eARGs in sewage and can be reused.

1. A method for removing extracellular antibiotic resistance genes in water, characterized by adding a cell lysate of bacteria to water, wherein the bacteria include one or more of E. coli, Acinetobacter sp., Pseudomonas putida and Acidithiobacillus ferrooxidans, the cell lysate contains enzymes that degrade DNA, and a temperature of the water is 28° C. to 37° C.

2. The method for removing extracellular antibiotic resistance genes in water according to claim 1, characterized in that the E. coli is E. coli MG1655, the Acinetobacter sp. is Acinetobacter baylyi ADP1, the Pseudomonas putida is Pseudomonas putida KT2440 and the Acidithiobacillus ferrooxidans is Acidithiobacillus ferrooxidans LX5.

3. The method for removing extracellular antibiotic resistance genes in water according to claim 1, characterized in that the cell lysate is immobilized by a hydrogel and then added to water.

4. The method for removing extracellular antibiotic resistance genes in water according to claim 3, characterized in that the hydrogel is a polyacrylamide hydrogel.

5. The method for removing extracellular antibiotic resistance genes in water according to claim 4, characterized in that a immobilization method of the hydrogel comprises obtaining a cell lysate, adding acrylamide, N,N′-bisacrylamide and K₂S₂O₈ to the cell lysate to obtain a mixture, using N₂ to aerate the mixture, and then performing a synthesis reaction of the hydrogel.

6. The method for removing extracellular antibiotic resistance genes in water according to claim 1, characterized in that the cell lysate is prepared by disrupting cells, wherein a method for disrupting cells is ultrasonic disruption, and conditions of the ultrasonic disruption are as follows: instrument parameters set to the power of 10% to 40%, a temperature of 0° C. to 4° C., an interval of 5.0 s to 9.9 s per running of 3.0 s to 5.0 s, and an ultrasonic time of 5 min to 10 min.

7. A microbial agent, characterized by comprising the cell lysate according to claim 1 or the immobilized cell lysate.

8. Use of the microbial agent according to claim 7 in the treatment of wastewater containing extracellular antibiotic resistance genes.
